(19) United States
(12) Patent Application Publication
(10) Pub. No.: US 2013/0225483 A1
(43) Pub. Date: Aug. 29, 2013

(54) New Treatments of Hepatitis C Virus Infection

(71) Applicants: Claudio Avila, Basel (CH); Rafael Crabbé, Bursins (CH); Nikolai Naoumov, Basel (CH)

(72) Inventors: Claudio Avila, Basel (CH); Rafael Crabbé, Bursins (CH); Nikolai Naoumov, Basel (CH)

(21) Appl. No.: 13/857,189

(22) PCT Filed: Oct. 3, 2011
(86) PCT No.: PCT/EP2011/067240

§ 371 Date: Apr. 5, 2013

(30) Foreign Application Priority Data
Oct. 5, 2010 (EP) 10186478.3

(51) Int. Cl. A61K 38/13

(52) U.S. Cl. CPC A61K 38/13
USPC 514/4.3; 530/321; 514/21.1

(57) ABSTRACT

The invention concerns the use of cyclophilin inhibitors in the treatment of Hepatitis C virus genotype 2 or 3 infection.

### BACKGROUND OF THE DISCLOSURE

The present disclosure relates to non-immunosuppressive cyclosporins which bind to cyclophilin, which are cyclophilin inhibitors, in particular to their pharmaceutical use of in the treatment of Hepatitis C virus infection.

The cyclosporins comprise a class of structurally distinctive, cyclic, poly-N-methylated undecapeptides, commonly possessing pharmacological, in particular immunosuppressive, or anti-inflammatory activity. The first of the cyclosporins to be isolated was the naturally occurring fungal metabolite Ciclosporin or Cyclosporine, also known as cyclosporin A (CsA).

Cyclosporins which bind strongly to cyclophilin but are not immunosuppressive have been identified. PCT/EP 2004/009804, WO 2005/021028, or WO 2006/071619 (which are incorporated by reference herein in their entirety) disclose non-immunosuppressive cyclosporins which bind to cyclophilin and have also been found to have an inhibitory effect on Hepatitis C virus (HCV). WO 2006/038088, incorporated herein by reference in its entirety, describes methods and compositions for the use of alisporivir in the treatment of HCV. Alisporivir (Debio-025 or DEB025 or DEB) is a cyclophilin (Cyp) inhibitor and its mode of action as an anti-HCV agent is via inhibition of host proteins, in particular of cyclophilin A, that are directly involved in HCV replication.

Hepatitis C virus (HCV) is an enveloped single stranded (+) RNA virus that belongs to the separate genus Hepacivirus of the family Flaviviridae. HCV causes acute and chronic liver disease, including chronic hepatitis, cirrhosis, and hepatocellular carcinoma. Worldwide more than 170 million people are chronically infected with HCV and are thus at increased risk of developing serious life-threatening liver disease.

The current standard of care in HCV genotypes 2 and 3 patients consists of a combination of interferon and ribavirin. Treatment duration and ribavirin dose depend on the genotype treated. Sustained viral response (SVR) in patients with HCV genotypes 2 and 3 after standard of care treatment reaches 80-90%, but side effects of standard of care treatment are significant and include myalgia, arthralgia, headache, fever, severe depression, leucopenia and haemolytic anaemia. These side effects are difficult to tolerate.

Persistent infection by HCV, which has been identified as the major causative agent of non-A, non-B hepatitis has been considered closely related to liver diseases such as chronic hepatitis, liver cirrhosis or hepatocellular carcinoma. The development of these liver diseases is a major public health problem.

Thus, despite existing therapies, there remains a significant need for methods and compositions for the treatment of HCV, safer, more tolerable therapies, more efficacious therapies, reliable alternatives for re-treatment if initial treatment fails.

### SUMMARY OF THE DISCLOSURE

Surprisingly we have determined that cyclophilin inhibitors, in particular alisporivir, can provide an effective alternative to the standard of care in treatment of HCV. In particular, we have found that efficacious treatment of Hepatitis C virus genotype 2 and 3 infection can be achieved when using alisporivir, avoiding the side effects of the current standard of care treatment and thus improving patient compliance. Furthermore, alisporivir can provide an efficacious treatment of Hepatitis C virus genotype 2 and 3 infection, with treatment duration of half the duration of the standard of care treatment.

Accordingly, the present disclosure provides new anti-HCV treatments using alisporivir, in particular methods of treating hepatitis C virus genotype 2 and 3 infection in a patient comprising administering to the patient alisporivir, during an initial phase in an amount of about 600 mg twice a day; followed by administering alisporivir during a second phase in an amount of at least about 600 mg, preferably of about 600 to about 1000 mg once per day.

The disclosure further provides alisporivir for use in the treatment or prevention of Hepatitis C virus genotype 2 and 3 infections or HCV induced disorders in patient.

Further, the following is described:

1.1 A method for preventing or treating Hepatitis C virus genotype 2 and 3 infections or HCV induced disorders in a patient, preferably a patient naive to treatment, comprising administering to said patient alisporivir during an initial phase in an amount of about 600 mg twice a day; followed by administering alisporivir during a second phase in an amount of at least about 600 mg, preferably of about 600 to about 1000 mg once per day.

1.2 A method for inhibiting HCV genotype 2 and 3 replication in a non-responder, comprising administering alisporivir during an initial phase in an amount of 600 mg twice a day; followed by administering alisporivir during a second phase in an amount of at least about 600 mg, preferably of about 600 to about 1000 mg once per day.

1.3 A method for preventing or delaying the recurrence of HCV genotype 2 and 3 infection in a transplant recipient, comprising administering to said recipient alisporivir during an initial phase in an amount of about 600 mg twice a day; followed by administering alisporivir during a second phase in an amount of at least about 600 mg, preferably of about 600 to about 1000 mg once per day.

1.4 A method for preventing or treating Hepatitis C virus genotype 2 and 3 infections or for preventing or treating HCV induced disorders in a patient, comprising administering to said patient alisporivir during an initial phase in an amount of about 600 mg twice a day; followed by administering alisporivir during a second phase in an amount of at least about 600 mg, preferably of about 600 to about 1000 mg once per day with an interferon and a ribavirin for a treatment duration of 12 weeks.

1.5 A method for preventing or treating Hepatitis C virus genotype 2 and 3 infections or for preventing or treating HCV induced disorders in a patient, comprising administering to said patient alisporivir during an initial phase in an amount of about 600 mg twice a day; followed by administering alisporivir during a second phase in an amount of at least about 600 mg, preferably of about 600 to about 1000 mg once per day for a treatment duration of 12 weeks.

2. Use of alisporivir in the preparation of a pharmaceutical composition for use in any method as defined above.

3. Use of alisporivir in the preparation of a medicament for use in any method as defined above.

4. A pharmaceutical composition for use in any method as defined above, comprising alisporivir, together with one or more pharmaceutically acceptable diluents or carriers therefor.

5. A therapeutic regimen comprising administering alisporivir during an initial phase in an amount of about 600 mg, twice per day followed by administering alisporivir during a second phase in an amount of at least about 600 mg, preferably of about 600 to about 1000 mg once per day; alisporivir may be administered in combination with ribavirin or with standard of care throughout the initial and second phases.

6. A package comprising the pharmaceutical composition comprising alisporivir as defined above, in combination with instructions to administer said composition during an initial phase in an amount of about 600 mg twice a day; followed by administering alisporivir during a second phase in an amount of at least about 600 mg, preferably of about 600 to about 1000 mg once per day.

7. A kit for the treatment of chronic hepatitis C virus genotype 2 and 3 infection.

Also contemplated herein are methods of reducing the HCV genotype 2 and 3 RNA in a patient comprising administering to the patient: alisporivir in which alisporivir is administered during an initial phase in an amount of about 600 mg twice a day; followed by administering alisporivir during a second phase in an amount of at least about 600 mg, preferably of about 600, about 800 or about 1000 mg once per day.

Additional embodiments of the present disclosure relate to methods of treating hepatitis C genotype 2 and 3 infections in a patient comprising administering to the patient: alisporivir, wherein alisporivir is administered during an initial phase in an amount of about 600 mg twice a day; followed by administering alisporivir during a second phase in an amount of at least about 600 mg, preferably of about 600 to about 1000 mg once per day.

Additional embodiments of the present disclosure relate to methods of treating hepatitis C genotype 2 and 3 infections in a patient comprising administering to the patient: alisporivir in combination with ribavirin, wherein alisporivir is administered during an initial phase in an amount of about 600 mg twice a day; followed by administering alisporivir during a second phase in an amount of about 600 to about 1000 mg once per day.

Additional embodiments of the present disclosure relate to methods of treating hepatitis C genotype 2 and 3 infections in a patient comprising administering to the patient: alisporivir in combination with interferon, wherein alisporivir is administered during an initial phase in an amount of about 600 mg twice a day; followed by administering alisporivir during a second phase in an amount of about 600 to about 1000 mg once per day. Also contemplated herein is a pharmaceutical combination comprising a first pharmaceutically acceptable formulation comprising alisporivir, a second pharmaceutically acceptable formulation comprising an interferon and a third pharmaceutically acceptable formulation comprising ribavirin, wherein the first, second and third formulations are packaged in a kit for the treatment of chronic hepatitis C virus genotype 2 and 3 infection.

### BRIEF DESCRIPTION OF THE FIGURE

FIG. 1 is a graph which illustrates the antiviral effect of the treatment with alisporivir (DEB) according to the present disclosure.

### DETAILED DESCRIPTION OF THE DISCLOSURE

Additional embodiments of the present disclosure relate to method for treating hepatitis C genotype 2 and 3 infections in a patient comprising administering alisporivir to such a level that the level of viral RNA in the patient decreases to an undetectable level and that a sustained viral response is achieved at the end of the treatment period.

In the above embodiments and throughout this specification, the standard of care treatment is a treatment that is used to treat Hepatitis C infections. The currently used standard of care treatment involves administration of an interferon, in particular pegylated interferon in combination with a ribavirin.

In the above embodiments and throughout this specification, the initial phase is a period of about 3, about 4, about 5, about 6, or about 7 days. Preferably the initial phase is a period of at least about 3 days, e.g., 3 days, more preferably about 7 days, e.g., 7 days.

In the above embodiments and throughout this specification, the second phase is a period of about 11, about 23, about 47 or about 71 weeks. Preferably the second phase is a period of about 23 weeks, e.g., 23 weeks.

In the above embodiments and throughout this specification, the treatment duration is the duration of the initial and second phases, provided that no other duration is specified.

In the present disclosure, the interferon may be pegylated or non-pegylated and may include interferons such as: Intron-A®, interferon alfa-2b (Schering Corporation, Kenilworth, N.J.); PEG-Intron®, peginteferon alfa-2b (Schering Corporation, Kenilworth, N.J.); Roferon®, recombinant interferon alfa-2a (Hoffmann-La Roche, Nutley, N.J.); Pegasys®, peginterferon alfa-2a (Hoffmann-La Roche, Nutley, N.J.); Berefor®, interferon alfa 2 available (Boehringer Ingelheim Pharmaceutical, Inc., Ridgefield, Conn.); Sumiferon®, a purified blend of natural alpha interferons (Sumitomo, Japan); Wellferon®, lymphoblastoid interferon alpha n1 (GlaxoSmithKline); Infergen®, consensus alpha interferon (InterMune Pharmaceuticals, Inc., Brisbane, Calif. and Amgen, Inc., Newbury Park, Calif.); Alferon®, a mixture of natural alpha interferons (Interferon Sciences, and Purdue Frederick Co., CT); Viraferon®; and combinations of these interferons.

Conjugated interferons that may be used include, for example, Joulferon®/Zalbin® (Albuferon®), albinterferon alfa 2b (Human Genome Sciences), which is conjugated to human albumin. Interferon conjugated to a water-soluble polymer or polyalkylene oxide homopolymers such as polyethylene glycol (PEG) or polypropylene glycols, polyoxyethylenated polyols, copolymers thereof and block copolymers thereof. As an alternative to polyalkylene oxide-based polymers, effectively non-antigenic materials such as dextran, polyvinyl pyrrolidones, polyacrylamides, polyvinyl alcohols, carbohydrate-based polymers and the like can be used. Interferon-polymer conjugates are described in U.S. Pat. No. 4,766,106, U.S. Pat. No. 4,917,888, EPA 0 236 987, EPA 0 510 356 and WO 95/13090. Since the polymeric modification sufficiently reduces antigenic responses, the foreign interferon need not be completely autologous. Interferon used to prepare polymer conjugates may be prepared from a mammalian extract, such as human, ruminant or bovine interferon, or recombinantly produced. Other forms of interferons include interferon beta, gamma, tau and omega, such as Rebif (Interferon beta 1a) by Serono, Omniferon (natural interferon) by Viragen, or Omega Interferon by Boehringer Ingelheim. Oral interferons such as oral interferon alpha by Amarillo Biosciences.

Additional examples of interferons that may be used include pegylated interferon alpha, for example pegylated interferon α-2a, pegylated interferon α-2b, pegylated consensus interferon or pegylated purified interferon-α product. Pegylated interferon α-2a is described in European Patent 593,868 (incorporated herein by reference in its entirety) and commercially available e.g. under the trade name PEGASYS® (Hoffmann-La Roche). Pegylated interferon-α-2b is described, e.g. in European Patent 975,369 (incorporated herein by reference in its entirety) and commercially available e.g. under the trade name PEG-INTRON A® (Schering Plough). Pegylated consensus interferon is described in WO 96/11953 (incorporated herein by reference in its entirety).

In preferred embodiments, the interferon used in the methods of the disclosure is pegylated interferon. In other embodiments, the interferon is selected from the group consisting of interferon alpha-2a, interferon alpha-2b, a consensus interferon, a purified interferon alpha product or a pegylated interferon alpha-2a, pegylated interferon alpha-2b, and pegylated consensus interferon, a mixture of natural alpha and combinations thereof.

Preferably the methods using interferon alpha use a pegylated interferon alpha-2b and the amount of pegylated interferon alpha-2b is from 0.5 to 2.0 micrograms/kilogram per week on a weekly, three times a week, every other day or daily basis.

As used herein, “microgram/kilogram” means microgram drug per kilogram body weight of the mammal—including man—to be treated.

As used herein, the term “treatment” or “treat” refer to both prophylactic or preventative treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment.

By “therapeutic regimen” is meant the pattern of treatment of an illness, e.g., the pattern of dosing used during HCV therapy. A therapeutic regimen may include an induction regimen and a maintenance regimen.

The phrase “initial phase” or “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both.

The phrase “second phase” or “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “about”, unless the context dictates otherwise, is used to mean a range of + or −10%.

In other embodiments, the interferon alpha is a pegylated interferon alpha-2a and the amount of pegylated interferon alpha-2a administered is from 20 to 250 micrograms/kilogram per week on a weekly, three times a week, every other day or daily basis. Preferably, the interferon peg-IFNa2a is administered at an amount of 180 ug once per week.

In specific embodiments, the exemplary interferon used in the methods herein is interferon selected from the group consisting of Intron-A®; PEG-Intron®; Roferon®; Pegasys®; Berefor®; Sumiferon®; Wellferon®; Infergen®; Alferon®; Viraferon®; Albuferon® (Human Genome Science); Rebif; Omniferon; Omega and combinations thereof.

In some embodiments, the patient may be administered ribavirin or a ribavirin derivative (e.g., a ribavirin analog or prodrug, such as ribamidine, taribavirin (viramidine), ICN 17261, molecules disclosed in WO/2008/052722, which is incorporated by reference in its entirety, etc.).

In some embodiments, ribavirin is administered at between about 800 to about 1200 mg per day, e.g., 1000 mg to 1200 mg per day. In some embodiments, ribavirin is administered based on the weight of the patient. In other embodiments, ribavirin is administered based on the HCV genotype of the patient.

In another embodiment, alisporivir may be administered with additional agents of the standard of care that promote the antiviral efficacy of the therapy treatment. The standard of care may include additional agents that promote the antiviral efficacy of the therapy treatment, such as substrate-based protease inhibitors of HCV NS3-4A serine protease, non-substrate-based NS3 protease inhibitors; phenanthrenequinones, thiazolidines and benzanilides, nucleosides analogs, antisense molecules directed against HCV genome or any cellular component that is required for viral replication, vaccine or antibody-based approaches to HCV treatment.

Direct acting antiviral agents, is used herein to mean agents that interfere with specific steps in the hepatitis C virus (HCV) replication cycle. Such agents may be, e.g., ribavirin derivatives, protease inhibitors, polymerase inhibitors (e.g., nucleoside and non-nucleoside inhibitors), and cyclophillin inhibitors. Exemplary direct acting antiviral agents include: boceprevir, telaprevir, ABT-072, ABT-450, ABT-333 by Abbott, ACH1625 by Achillion, ANA598 by Anadys Pharmaceuticals, AZD-7295 by AstraZeneca, BI201335, BI207127 by Boehringer Ingelheim Pharma, BMS650032, BMS790052, BMS791325, BMS824383 by Bristol Myers Squibb, Clemizole by Eiger BioPharmacetucials, Filibuvir by Pfizer, GS9190 (Tegobuvir), GS9256 by Gilead, IDX375 by Idenix, INX-189 by Inhibitex, PSI-7851, PSI-938 by Pharmasset, PSI-7977, RG7128 by Pharmasset/Genethec, PPI-461 by Presidio RG7227 (Danoprevir) by InterMune/Genentech, SCH900518 (Narlaprevir), Vaniprevir by Merck, TMC435 by Medivir/Tibotec, VX-222, VX-759, VX-500, VX-916 by Vertex.

As used herein “up to 12, 24, 48 or 72 weeks” refers to the treatment duration and is intended to mean for about 12 weeks, about 24 weeks, about 48 weeks, or about 72 weeks, respectively. It will be understood that therapy need not end at exactly the 12, 24, 48 or 72 week time period. For example, therapy may end a day or a few days before the 24 week period, and still be an equivalent within the scope and spirit of the current disclosure.

In one embodiment, the present disclosure further provides alisporivir for use in combination with standard of care in treatment of a Hepatitis C virus genotype 2 and 3 infected patient, the alisporivir being administered during an initial phase in an amount of about 600 mg, twice per day; followed by administering alisporivir during a second phase in an amount of at least about 600 mg, preferably of about 600 to about 1000 mg once per day. In still another aspect, the initial phase is a period of about 7 days; the second phase is a period of about 11, about 23 or about 47 weeks.

In one embodiment, the present disclosure further provides alisporivir for use in treatment of a Hepatitis C virus genotype 2 or 3 infected patient characterized in that (i) alisporivir is administered during an initial phase in an amount of about 600 mg, twice per day; (ii) followed by administering alisporivir during a second phase in an amount of at least about 600 mg once per day.

In one embodiment, the present disclosure further provides alisporivir for use in treatment of a Hepatitis C virus genotype 2 or 3 infected patient characterized in that (i) alisporivir is administered during an initial phase in an amount of about 600 mg, twice per day; (ii) followed by administering alisporivir during the second phase in an amount of about 600 mg to about 1000 mg once per day.

In one embodiment, the present disclosure further provides alisporivir for use in treatment of a Hepatitis C virus genotype 2 or 3 infected patient characterized in that (i) alisporivir is administered during an initial phase in an amount of about 600 mg, twice per day; (ii) followed by administering alisporivir during the second phase in an amount of 600 mg or about 800 mg once per day and wherein alisporivir is administered in combination with ribavirin throughout the initial and second phases.

In one embodiment, the present disclosure further provides alisporivir for use in treatment of a Hepatitis C virus genotype 2 or 3 infected patient characterized in that (i) alisporivir is administered during an initial phase in an amount of about 600 mg, twice per day; (ii) followed by administering alisporivir during the second phase in an amount of 600 mg once per day and wherein alisporivir is administered in combination with interferon throughout the initial and second phases.

In one embodiment, the present disclosure further provides alisporivir for use in treatment of a Hepatitis C virus genotype 2 or 3 infected patient characterized in that (i) alisporivir is administered in the initial phase at an amount of 600 mg twice a day for 7 days; (ii) followed by administering alisporivir in the second phase in an amount of about 600 mg or 800 mg once daily for 3 weeks or 5 weeks or 7 weeks, and (iii) if HCV RNA in the patient plasma is detectable by a HCV-RNA assay following step (ii), then administering alisporivir in an amount of 600 mg once a day in combination with standard of care for up to 12 or 24 weeks.

In one embodiment, the present disclosure further provides alisporivir for use in treatment of a Hepatitis C virus genotype 2 or 3 infected patient characterized in that (i) alisporivir is administered in the initial phase in an amount of 600 mg twice a day for 7 days, in combination with ribavirin; (ii) followed by administering alisporivir in the second phase in an amount of about 600 mg once daily for 3 weeks, and (iii) if the HCV RNA is detectable by a HCV-RNA assay following step (ii), then administering alisporivir in an amount of 600 mg once a day in combination with ribavirin and an interferon for up to 12 or 24 weeks.

In one embodiment, the present disclosure further provides alisporivir for use in combination with an interferon and/or a ribavirin in treatment of a Hepatitis C virus genotype 2 or 3 infected patient, the alisporivir being administered during an initial phase in an amount of about 600 mg, twice per day for about 7 days; followed by administering alisporivir during a second phase in an amount of about 600, about 800 or about 1000 mg once per day for up to about 11 or about 23 weeks.

In one embodiment, the present disclosure further provides alisporivir for use in combination with ribavirin in treatment of a Hepatitis C virus genotype 2 or 3 infected patient, the alisporivir being administered during an initial phase in an amount of about 600 mg, twice per day for about 7 days; followed by administering alisporivir during a second phase in an amount of about 600, about 800 or about 1000 mg once per day for up to about 23 weeks.

In one embodiment, the present disclosure further provides alisporivir for use in combination with interferon in treatment of a Hepatitis C virus genotype 2 or 3 infected patient, the alisporivir being administered during an initial phase in an amount of about 600 mg, twice per day for about 7 days; followed by administering alisporivir during a second phase in an amount of about 600, about 800 or about 1000 mg once per day for up to about 23 weeks.

In one embodiment, the present disclosure further provides alisporivir for use in treatment of a Hepatitis C virus genotype 2 or 3 infected patient, the alisporivir being administered during an initial phase in an amount of about 600 mg, twice per day for about 7 days; followed by administering alisporivir during a second phase in an amount of 1000 mg once per day for up to up to about 47 weeks, most preferred up to about 23 weeks.

In one embodiment, the present disclosure further provides alisporivir for use in combination with ribavirin in treatment of a Hepatitis C virus genotype 2 or 3 infected patient, the alisporivir being administered during an initial phase in an amount of about 600 mg, twice per day for about 7 days; followed by administering alisporivir during a second phase in an amount of 600 mg once per day for up to up to about 47 weeks, most preferred up to about 23 weeks.

In one embodiment, the present disclosure further provides alisporivir for use in combination with interferon in treatment of a Hepatitis C virus genotype 2 or 3 infected patient, the alisporivir being administered during an initial phase in an amount of about 600 mg, twice per day for about 7 days; followed by administering alisporivir during a second phase in an amount of 800 mg once per day for up to up to about 47 weeks, most preferred up to about 23 weeks.

In one embodiment, the present disclosure further provides alisporivir for use in treatment of a Hepatitis C virus genotype 2 or 3 infected patient, the alisporivir being administered during an initial phase in an amount of about 600 mg, twice per day for about 7 days; followed by administering alisporivir during a second phase in an amount of 1000 mg once per day for up to about 23 weeks.

In one embodiment, the present disclosure further provides alisporivir for use in combination with pegylated interferon alpha-2a in treatment of a Hepatitis C virus genotype 2 or 3 infected patient, alisporivir being administered during an initial phase in an amount of about 600 mg, twice per day; followed by administering alisporivir during a second phase in an amount of about 800 mg once per day for up to about 11, about 23 or about 47 weeks. In still another aspect, the pegylated interferon alpha-2a is administered in an amount of 180 micrograms once per week.

In one embodiment, the present disclosure further provides alisporivir for use in combination with ribavirin in treatment of a Hepatitis C virus genotype 2 or 3 infected patient, the alisporivir being administered during an initial phase in an amount of about 600 mg, twice per day; followed by administering alisporivir during a second phase in an amount of about 800 mg once per day for up to about 11, about 23 or about 47 weeks. In still another aspect, the ribavirin is administered at between 1000 mg to 1200 mg per day.

In one embodiment, the present disclosure further provides alisporivir for any use as defined above, wherein if after four weeks of treatment the HCV RNA is detectable by a HCV-RNA assay then administering alisporivir in an amount of 600 mg once a day in combination with standard of care or with ribavirin for up to the end of the treatment duration.

In one embodiment, the present disclosure further provides alisporivir for use in treatment of a Hepatits C virus genotype 2 or 3 infected patient, wherein alisporivir is administered during an initial phase in an amount of 600 mg twice a day followed by administering alisporivir 1000 mg once daily for up to about 4 weeks, and if the HCV RNA is detectable by a HCV-RNA assay, then administering alisporivir in an amount of 600 mg once a day in combination with standard of care until the end of the treatment. In still another aspect, the initial phase is a period of about 7 days; the treatment duration is of about 12, about 24 or about 48 weeks.

In one embodiment, the present disclosure further provides alisporivir for use in treatment of a Hepatitis C virus genotype 2 or 3 infected patient, wherein alisporivir is administered in combination with ribavirin, in an amount of 600 mg twice a day for 7 days, followed by 600 mg administered once daily, for 3 weeks, and if the HCV RNA is detectable by a HCV-RNA assay then administering alisporivir in an amount of 600 mg once a day in combination with standard of care for up to 24 weeks.

In one aspect, the present disclosure further provides a method of treating of a Hepatitis C virus genotype 2 or 3 infected patient with alisporivir in combination with ribavirin, the method comprising administering alisporivir during an initial phase in an amount of about 600 mg, twice per day; followed by administering alisporivir during a second phase in an amount of about at least 600 mg, preferably of about 600 to about 1000 mg once per day for up to about 11, about 23 or about 47 weeks. In still other aspects, the initial phase is a period of at least about 3 days, preferably of about 5 days, most preferred of about 7 days.

In one aspect, the present disclosure further provides use of alisporivir in the manufacture of a medicament for treatment of a Hepatitis C virus genotype 2 or 3 infected patient wherein alisporivir is administered during an initial phase in an amount of about 600 mg, twice per day; followed by administering alisporivir during a second phase in an amount of about at least 600 mg, preferably of about 600 to about 1000 mg once per day for up about 11, about 23 or about 48 weeks and wherein alisporivir may be administered in combination with interferon or ribavirin throughout the initial and second phases. In still other aspects, the initial phase a period of at least about 3 days, preferably of about 5 days, most preferred of about 7 days.

In one aspect, the present disclosure further provides use of alisporivir in the preparation of a pharmaceutical composition for treatment of a Hepatitis C virus genotype 2 or 3 infected patient characterized in that alisporivir is administered during an initial phase in an amount of about 600 mg, twice per day; followed by administering alisporivir during a second phase in an amount of about at least 600 mg, preferably of about 600 to about 1000 mg once per day for up to about 11, about 23 or about 47 weeks and wherein alisporivir is administered in combination with interferon or ribavirin throughout the initial and second phases. In still other aspects, the initial phase is a period of at least about 3 days, preferably of about 5 days, most preferred of about 7 days.

In one aspect, the present disclosure further provides a combination of alisporivir with interferon or ribavirin for use in treatment of a Hepatitis C virus genotype 2 or 3 infected patient, wherein alisporivir is administered during an initial phase in an amount of about 600 mg, twice per day for about 7 days; followed by administering alisporivir during a second phase in an amount of about 600 to about 1000 mg once per day for up to about 11, about 23 or about 47 weeks.

In one aspect, the present disclosure further provides a therapeutic regimen comprising administering alisporivir during an initial phase in an amount of about 600 mg, twice per day for one week; followed by administering alisporivir during a second phase in an amount of about 600 to about 1000 mg once per day for up to about 23, or about 47 and wherein alisporivir is administered in combination with interferon or ribavirin throughout the initial and second phases.

In one aspect, the present disclosure further provides pharmaceutical compositions comprising alisporivir for uses as defined above. In still other aspects, the present disclosure provides a package comprising the pharmaceutical composition comprising alisporivir for uses as defined above in combination with instructions to administer said composition.

In one aspect, the present disclosure further provides a use of alisporivir for the manufacture of a medicament for the treatment of a Hepatitis C virus genotype 2 or 3 infected patient wherein the medicament is formulated at a dosage comprising one, two, three, four or five dose of about 50 mg to about 200 mg. In still other aspects, the present disclosure provides use of alisporivir for the manufacture of a medicament for the treatment of a Hepatitis C virus genotype 2 or 3 infected patient wherein the medicament is formulated at a dosage comprising one, two, three or four dose of about 100 mg or about 200 mg. In still other aspects, the medicament is formulated at a dosage comprising one, two, three, four or five dose of about 50 mg to about 200 mg, further comprising ribavirin.

In one aspect, the present disclosure further provides use of alisporivir for the manufacture of a medicament for the treatment of a Hepatitis C virus genotype 2 or 3 infected patient wherein the medicament is formulated at a dosage comprising one, two, three, four or five dose of about 50 mg to about 200 mg, further comprising ribavirin, wherein the content of ribavirin in the medicament is at between about 800 to about 1200 mg per day, e.g., 1000 mg to 1200 mg per day mg per dosage unit.

In one aspect, the present disclosure further provides a pharmaceutical composition comprising alisporivir for any use as defined above.

In one aspect, the present disclosure further provides a kit comprising

a) a pharmaceutical composition comprising alisporivir for use in the treatment of a Hepatitis C virus genotype 2 or 3 infected patient, optionally in combination with one or more pharmaceutically acceptable excipients, and

b) instructions describing how to administer said pharmaceutical composition for the treatment of a Hepatitis C virus genotype 2 or 3 infected patient, wherein the administration is characterized by
(i) alisporivir is to be administered in the initial phase at an amount of 600 mg twice a day for 7 days;
(ii) followed by administering alisporivir in the second phase in an amount of about 1000 mg once daily for 3 weeks, and
(iii) if the HCV RNA is detectable by a HCV-RNA assay following step (ii), then alisporivir is to be administered in an amount of 600 mg once a day in combination with standard of care for up to 12 or 24 weeks.

In exemplary embodiments, alisporivir is administered at a dosage of from about 600 to about 1000 mg twice daily for about 7 days followed by administering alisporivir at a dosage of from about 600 to about 1000 mg once per day for up to about 11, about 23 or about 47 weeks.

In exemplary embodiments, the treatment of the present disclosure involves administration of interferon alpha that is a pegylated interferon alpha-2a and the amount of pegylated interferon alpha-2a administered is from 20 to 250 micrograms per week on a weekly, three times a week, every other day or daily basis. The current approved dose is 180 micrograms per week. In other exemplary embodiments, the interferon alpha is a pegylated interferon alpha-2b and the amount of pegylated interferon alpha-2b is from 0.5 to 2.0 micrograms/kilogram per week on a weekly, three times a week, every other day or daily basis. Exemplary descriptions of such treatments are described in U.S. Pat. No. 7,115,578, incorporated herein by reference in its entirety.

An exemplary Peg-IFNα2a used in the treatment protocols described herein is Pegasys®. PEGASYS® is a pegylated form of IFNα2a (peg-IFNα2a or PegINF) and utilizes a 40 kDa branched PEG (polyethylene glycol) to provide sustained serum concentrations for a full week (168 hours). PEGASYS® is commercially available, presented as single use, pre-filled syringes containing 180 mg/0.5 mL peg-IFNα2a for S.C. injection. The standard package contains 1 syringe of 180 μg/0.5 mL.

In some embodiments, it may be desirable to modify the dose of Peg-IFNα2a. If dose modification is required for moderate to severe adverse reactions (clinical and/or laboratory), initial dose reduction from 180 to 135 mg is generally adequate (adjustment to the corresponding graduation mark on pre-filled syringe). However, in some cases, dose reduction to 90 mg may be needed. Following improvement, re-escalation of the dose may be considered.

In treatment described above effective dosages of the standard of care agents are administered in compositions, i.e. they may be administered together (i.e., simultaneously), but may also be administered separately or sequentially. In general, combination therapy is typically administered together, the rationale being that such simultaneous administration induces multiple simultaneous stresses on the virus. The specific dosages given will depend on absorption, inactivation and excretion rate of the drugs as well as other factors. It is to be noted that dosage values will also vary with the severity of the condition to be alleviated.

The terms “co-administration” or “combined administration” or “administered in combination with” or the like as utilized herein are meant to encompass administration of the selected therapeutic agents to a single patient, and are intended to include treatment regimens in which the agents are not necessarily administered by the same route of administration or at the same time. Fixed combinations are also within the scope of the present disclosure. The administration of a pharmaceutical combination of the disclosure results in a beneficial effect, e.g. a synergistic or additive therapeutic effect, compared to a monotherapy applying only one of its pharmaceutically active ingredients or as compared to the current standard of care therapy. The treatment used in the methods described herein may be administered by any conventional route. One or more components may be administered parentally, e.g., in the form of injectable solutions or suspensions, or in the form of injectable deposit formulations. Preferably, alisporivir will be administered orally in the form of solutions or suspensions for drinking, tablets or capsules. Pharmaceutical compositions for oral administration comprising alisporivir typically further comprise one or more pharmaceutically acceptable carrier substances.

Typically, these compositions are concentrated and need to be combined with an appropriate diluent, e.g., water, prior to administration. Pharmaceutical compositions for parenteral administration typically also include one or more excipients. Optional excipients include an isotonic agent, a buffer or other pH-controlling agent, and a preservative. These excipients may be added for maintenance of the composition and for the attainment of preferred ranges of pH (about 6.5-7.5) and osmolarity (about 300 mosm/L).

The administration of alisporivir as described herein is in a single dose form or in more than one dosage form; one or more oral dosage forms may be administered at each time per day. In some embodiments, alisporivir is administered in doses of 200 mg to 1000 mg.

In the present application, the term “non-responder” is intended to mean a patient or subject who is a non-responder to standard of care treatment for HCV. More specifically, a non-responder to standard of care patient is a patient who has not responded to treatment with standard of care given over a 12 weeks treatment period. The non-responder to standard of care includes the following subsets of patients—null responders and partial responders.

Typically, a patient who has a “null response” may, for example, be defined as one in whom the HCV-RNA reduction is observed to be less than 2 log 10 IU/mL after 12 weeks of treatment with standard of care.

A patient that has a “partial” response or partial responder is one in whom the HCV-RNA reduction of more than 2 log 10 IU/mL is observed after 12 weeks of treatment with standard of care but the HCV-RNA is still detectable at the end of treatment.

The efficacy of the therapy regimen may be monitored using standard protocols. Treatment may be followed by determinations of HCV in serum and measurement of serum ALT levels. For example, the patients may be assessed for the presence of HCV RNA in their plasma. HCV RNA (IU/mL) can be measured at regular intervals during the treatment, e.g., at Day 1 (pre-dose and 4, 8, and 12 hours post-dose) and pre-dose at Day 2, Day 3, Day 8, Day 15, Day 29, and at Week 12, Week 24, Week 36, Week 48, Week 72 (when applicable), and at follow up. In addition, the HCV strains in the patient can be sequenced and assessed for identification of mutations selecting for resistance.

As used herein, LOD means limit of detection (serum HCV RNA is less than 10 IU/mL) and LOQ means limit of quantification (serum HCV RNA is less than 25 IU/mL). HCV RNA levels can be measured using commercially available methods.

The endpoint of treatment is a virological response, i.e., the absence of HCV at the end of a treatment course, several months after initiation of treatment, or several months after completion of treatment. HCV in serum may be measured at the RNA level by methods such as quantitative RT-PCR or northern blots or at the protein level by enzyme immunoassay or enhanced chemiluminescence immunoassay of viral proteins. The endpoint may also include a determination of a serum ALT level in the normal range.

The virological response parameters are: rapid virologic response at treatment week 4 (RVR 4) defined by undetectable serum HCV-RNA at treatment week 4; early virological response (EVR), defined by at least 2 log 10 IU/mL reduction in HCV-RNA compared to baseline (partial EVR) or undetectable serum HCV-RNA (complete EVR) at treatment week 12; sustained virological response (SVR24), defined as absence of HCV-RNA from serum by a sensitive Polymerase Chain Reaction (PCR) assay 24 weeks following end of therapy or the HCV RNA is undetectable (by LOD) 24 weeks after end of treatment; End of Treatment Response (ETR): HCV RNA undetectable (by LOD) at treatment end (completed or prematurely discontinued).

Exemplary therapeutic regimens are given in the Examples. In one exemplary protocol a subject in need of treatment is provided with 600 mg alisporivir orally twice daily for 7 days, followed by 1000 mg alisporivir orally once daily for 23 weeks.

In another exemplary therapeutic regimen a subject in need of treatment is provided with ribavirin administered in an oral dosage of 1000/1200 mg daily (weight based) for 24 weeks in combination with 600 mg alisporivir orally twice daily for 7 days, followed by 600 mg alisporivir orally once daily for 23 weeks.

In yet another exemplary therapeutic regimen a subject in need of treatment is provided with pegylated interferon alfa 2a at a dose of 180 mg subcutaneously (S.C.) once weekly for 24 weeks, in combination with 600 mg alisporivir orally twice daily for 7 days, followed by 800 mg alisporivir orally once daily for 23 weeks.

After a 4 week treatment period, based on patient response, the administration of alisporivir may be continued up to 24 weeks from the start of treatment at 600 mg with pegylated interferon alfa 2a and ribavirin. For example between weeks 5 to 24, the patient is administered, in addition to alisporivir as defined above, 180 μg pegylated interferon alfa 2a S.C. orally once weekly and ribavirin administered in an oral dosage of 1000/1200 mg daily (weight based).

The following Examples illustrate the embodiments of the disclosure described hereinbefore.

### Examples

1. Compounds

Peg-IFNα2a is a pegylated form of interferon alfa 2a and utilizes 40 kDa branched PEG (polyethylene glycol) to provide sustained serum concentrations for a full week (168 hours). PEGASYS® is commercially available from Roche.

Ribavirin is a synthetic nucleoside analogue and is also commercially available, e.g., as COPEGUS® from Roche.

2. Clinical Study and Results

An international, multicentre, randomized, parallel groups, open-label, multiple dose phase II study.

Patients are randomized into one of 5 treatment arms (A, B, C, D and E) as described below in a 2:2:2:1:1 ratio.

Treatment A

Alisporivir: 3 capsules of 200 mg (600 mg) alisporivir 2×/day (twice a day or BID) orally for 1 week followed by 5 capsules of 200 mg alisporivir (1000 mg) 1×/day (once a day or QD) for 23 weeks.

Treatment B

Alisporivir: 3 capsules of 200 mg (600 mg) alisporivir 2×/day orally for 1 week followed by 3 capsules alisporivir (600 mg) 1×/day for 23 weeks.

Ribavirin: 400 mg, 2×/day for 24 weeks

Treatment C

Alisporivir: 3 capsules of 200 mg (600 mg) alisporivir 2×/day orally for 1 week followed by 4 capsules alisporivir (800 mg) lx/day for 23 weeks.

Ribavirin: 400 mg, 2×/day for 24 weeks

Treatment D

Alisporivir: 3 capsules of 200 mg (600 mg) alisporivir 2×/day orally for 1 week followed by 3 capsules alisporivir (600 mg) 1×/day for 23 weeks.

Pegylated interferon alfa 2a: 180 μg subcutaneously (s.c.) once weekly for 24 weeks

Treatment E

Ribavirin: 400 mg, 2×/day for 24 weeks

Pegylated interferon alfa 2a: 180 μg subcutaneously (s.c.) once weekly for 24 weeks

Primary efficacy endpoint: proportion of patients achieving RVR (rapid viral response) after 4 week treatment as described above.

Patients in treatment arms A, B, C, and D who have HCV RNA levels above LOQ (25 IU/mL) at treatment week 4 are to continue with triple therapy from Week 6 (which is the next visit), therefore, all patients remain on the initial treatment regimen for up to 6 weeks. From week 6, patients who had not achieved RVR at week 4 received alisporivir 600 mg in combination with peg-IFNα2a and ribavirin (alisporivir 600 mg+peg-IFNα2a/RBV).

This study was designed to explore the potential of alisporivir-based interferon-free treatment in previously untreated HCV genotype 2 (GT2) and genotype 3 (GT3) patients.

A total of 340 patients were randomized. Week 4 interim analysis only included 334 patients. The number of patients in randomized set, full analysis set and safety set was the same for each treatment group.

The mean pre-treatment HCV RNA levels were 6.00 Log 10 IU/mL with standard deviation±1.00 Log 10 IU/mL.

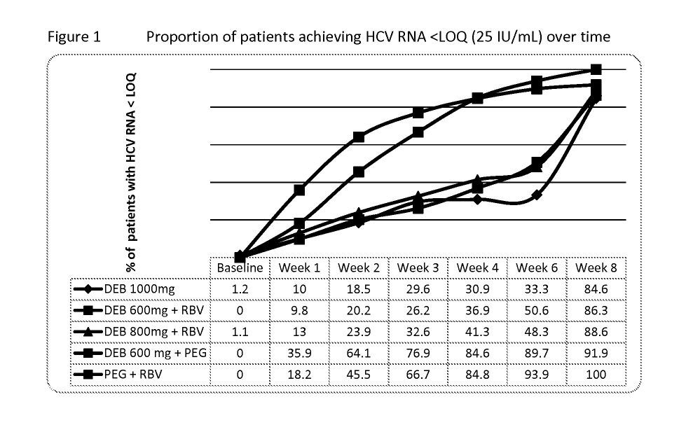

FIG. 1 presents the proportion of patients (expressed in percentage) achieving HCV RNA negative by LOQ from baseline up to Week 8, when treated according to the above clinical trial protocol.

Approximately 50% of the patients on the interferon-free alisporivir in combination with ribavirin arms achieved HCV RNA negative results by week 6 of the treatment. In the alisporivir monotherapy arm, one third of patients achieved HCV RNA negative results.

According to the study design, patients who were HCV RNA positive at week 4 (HCV RNA>LOQ, 25 IU/mL) continued on their initial treatment until week 6, when they received alisporivir 600 mg QD+PegIFN/RBV, or if they were HCV RNA negative (HCV RNA<LOQ, 25 IU/mL) at week 4, they remained on their initial dual therapy treatment regimen after week 6.

In the alisporivir-based interferon-free treatment arms, intensification to triple therapy from week 6 (alisporivir+PegIFN+RBV) for those patients who did not achieve HCV RNA negative levels at week 4, resulted in a sharp increase in the proportion of patients achieving HCV RNA negative results at week 8, reaching similar HCV RNA negative levels to those receiving the interferon based treatments from baseline.

The proportion of HCV RNA negative patients increased over time for all treatment groups. Up until week 6, at most time points, the two interferon-free dual therapy groups with alisporivir and RBV achieved a numerically higher proportion of HCV RNA negative patients than the alisporivir monotherapy group, while the treatment groups with interferon had greater proportion of HCV RNA negative patients compared to the interferon-free treatment groups.

The proportion of HCV RNA negative patients in the interferon-free treatment groups increased markedly once triple therapy with alisporivir+PegIFN/RBV was given, for those who had not achieved RVR4LOQ. Within only 2 weeks, the proportion of HCV RNA negative patients reached similar levels to those on interferon containing treatment from baseline.

Approximately 30% of patients were HCV G2, as planned. Overall, the proportion of HCV RNA negative patients was similar between G2 and G3 patients over the 8 weeks of treatment.

Overall the alisporivir-based treatment regimens were well tolerated, with low rates of discontinuation due to adverse events. Overall, interferon-free arms had less adverse events than interferon-containing arms.

Expected interferon-related symptoms (fatigue, pyrexia, asthenia, chills, influenza like illness decreased appetite, myalgia, arthralgia, headache and pruritis) were experienced frequently in both interferon arms, but not in the alisporivir interferon-free arms. Alisporivir once daily shows promise to become the first IFN-free oral treatment for a substantial proportion of G2/3 patients.

1. Alisporivir for use in treatment of a Hepatitis C virus genotype 2 or 3 infected patient characterized in that (i) alisporivir is administered during an initial phase in an amount of about 600 mg, twice per day; (ii) followed by administering alisporivir during a second phase in an amount of at least about 600 mg once per day.

2. Alisporivir for use according to claim 1 wherein alisporivir is administered during the second phase in an amount of about 600 mg to about 1000 mg once per day.

3. Alisporivir for use according to claim 1 wherein the alisporivir is administered during the second phase in an amount of 600 mg or about 800 mg once per day and wherein alisporivir is administered in combination with ribavirin or standard of care throughout the initial and second phases, for a treatment duration of 12 weeks.

4. Alisporivir for use according to claim 3 wherein the patient is a non-responder patient.

5. Alisporivir for use according to claim 1 wherein
(i) alisporivir is administered in the initial phase in an amount of 600 mg twice a day for 7 days, in combination with ribavirin; (ii) followed by administering alisporivir in the second phase in an amount of about 600 mg or 800 mg once daily for 3 weeks, for 5 weeks or for 7 weeks, in combination with ribavirin and
(iii) if the HCV RNA in patient plasma is detectable by a HCV-RNA assay following step (ii), then alisporivir is to be administered in an amount of 600 mg once a day in combination with ribavirin and interferon for up to 24 weeks.

6. A method of treating a Hepatits C virus genotype 2 or 3 infected patient, comprising administering alisporivir during an initial phase in an amount of about 600 mg, twice per day; followed by administering alisporivir during a second phase in an amount of at least about 600 mg once per day.

7. The method according to claim 6 wherein alisporivir is administered during the second phase in an amount of about 600 mg or about 800 mg once per day for about 23 weeks, in combination with ribavirin throughout the initial and second phases.

8. A therapeutic regimen comprising, administering alisporivir during an initial phase in an amount of about 600 mg, twice per day for one week; followed by administering alisporivir during a second phase in an amount of at least about 600 mg once per day.

9. The therapeutic regimen according to claim 8, wherein alisporivir is administered during the second phase in an amount of about 600 mg once per day afor about 23 weeks, and wherein said alisporivir is administered in combination with ribavirin throughout the initial and second phases.

10. The therapeutic regimen according to claim 8, wherein alisporivir is administered during the second phase in an amount of about 800 mg once per day for about 23 weeks, and wherein said alisporivir is administered in combination with ribavirin throughout the initial and second phases.

11. A pharmaceutical composition comprising alisporivir for use according to claim 1.

12. A package comprising the pharmaceutical composition according to claim 11 in combination with instructions to administer said composition.
